(19) United States
(12) Patent Application Publication
(10) Pub. No.: US 2003/0119205 A1
(43) Pub. Date: Jun. 26, 2003

(54) Method for rapid catalyst screening

(75) Inventor: John Patrick Lemmon, Delanson, NY (US)

Correspondence Address: JEFFREY S. BERNARD, DOUGHERTY, CLEMENTS & HOFER, 1901 ROXBOROUGH ROAD, SUITE 300, CHARLOTTE, NC, 28211, US

(73) Assignee: General Electric Company, Niskayuna, NY

(21) Appl. No.: 09/683,416

(22) Filed: Dec. 24, 2001

(51) Int. Cl. G01N 31/00; G01N 33/00; G01N 33/543

(52) U.S. Cl. 436/518; 436/34

(57) ABSTRACT

The present invention provides methodology for the rapid discovery of catalytically-active species. In the method of the invention, gradient arrays of catalytic species to be evaluated are absorbed on or impregnated into a support material. Next, the supported array is placed in a single reactor and the desired chemical reaction is carried out. The reaction products stream is then analyzed for the existence of the desired reaction products. If desired reaction products are observed to be present, the support library is divided at least in one half and the reaction conducted again. This technique is then repeated until a single catalyst mixture (or multiple catalyst mixtures) is identified as having the desired catalytic activity.

### FEDERAL RESEARCH STATEMENT

[This invention was made with government support under Contract No. DEFC0298CH10931 awarded by the Department of Energy. The government may have certain rights to the invention.]

### BACKGROUND OF INVENTION

The present invention is directed to a method for rapid screening of inorganic materials for potential catalytic activity. In particular, the invention relates to a method for preparing combinatorial libraries of supported catalysts, and the screening of such catalysts in a single reactor.

Since its introduction in 1970, combinatorial chemistry has become a popular research tool among scientists in many fields. High throughput and combinatorial screening for biological activity has been prevalent in the pharmaceutical industry for nearly twenty years, and more recently, high throughput and combinatorial screening for improved catalysts for the bulk chemical industry has enjoyed increasing popularity.

In the art of catalysis, there is sometimes little predictability between the composition and/or structure of a material and its catalytic properties, due to a lack of information. Therefore, essentially the best way to determine if a particular material is a good catalyst is by testing the material under actual conditions encountered in the process of interest. When it is desired to discover a new catalyst useful for a particular reaction, large groups of different materials are each individually tested and those failing to show a predetermined minimal activity for the conversion of the starting material to the desired end product are screened out.

A substantial reason for the lag in the development of high throughput and combinatorial screening for production scale reactions is the difficulty in emulating the production-scale reactions at the micro-scale necessary for high throughput or combinatorial work.

Most combinatorial work to date has focused on “solid phase” reactions. It is known that a wide variety of organic reactions can be carried out on substrates immobilized on resins. However, a substantial number of production scale reactions are “liquid phase” or “mixed phase” and, as noted, are carried out in continuous flow reactor systems.

Early efforts in high throughput screening of solutions have focused on catalyst screening. Before the application of the high throughput and combinatorial approaches, catalyst testing was traditionally accomplished in bench scale units or larger pilot plants in which the feed to a continuous flow reactor was contacted with a catalyst under near steady state reaction conditions. However, rapid and combinatorial screening of reactants, catalysts, and associated process conditions require that a large number of reactions or catalytic systems be tested simultaneously. In certain applications, screening-level data can be generated by using miniaturized batch reactors in conjunction with liquid-handling robots that aliquot the appropriate catalysts and reactants to each vial or reaction well. In other applications, however, batch reactions do not behave in the same fashion as continuous flow reactions and could provide misleading results if the goal of screening is to identify reactants or catalyst systems that will be implemented in production-scale continuous flow reactors.

As the demand for bulk chemicals has continued to grow, new and improved methods of producing more product with existing resources are needed to supply the market. Unfortunately, the identities of additional effective reactants and catalyst systems for these processes continue to elude the industry. What are needed are new and improved methodologies for rapid screening of catalysts.

### SUMMARY OF INVENTION

The present invention provides methodology for the rapid discovery of catalytically-active species. In the method of the invention, gradient arrays of inorganic species to be evaluated are adsorbed onto, impregnated into a support material, or otherwise attached to a support material. Next, the supported array is placed in a single reactor and the desired chemical reaction is carried out. The reaction products stream is then analyzed for the existence of the desired reaction products. If desired reaction products are observed to be present, the support library is divided at least in one half and the reaction conducted again. This technique is then repeated until a single catalyst mixture (or multiple catalyst mixtures) is identified as having the desired catalytic activity. When larger pellets are utilized as the support media, gross physical characteristics can be analyzed in order to provide a rough qualitative evaluation of whether the pellet contained catalytically active species. For example, if the pellet were darkly colored, this may indicate the existence of organic material, thus evidencing an oxidative decomposition reaction rather than a successful catalysis.

### BRIEF DESCRIPTION OF DRAWINGS

FIG. 1 is a diagram illustrating a ternary combinatorial array format.

FIG. 2 is a functional block diagram illustrating the high throughput doping of a catalyst support material.

FIG. 3 is a diagram of a glass reactor used in carrying out the reactions of the present invention.

### DETAILED DESCRIPTION

As required, detailed embodiments of the present invention are disclosed herein; however, it is to be understood that the disclosed embodiments are merely exemplary of the invention that may be embodied in various and alternative forms. Specific structural and functional details disclosed herein are not to be interpreted as limiting, but merely as a basis for the claims and as a representative basis for teaching one skilled in the art to variously employ the present invention.

The present invention provides a method for the rapid screening of catalytically active species, which includes the steps: (a) providing a gradient array of potential inorganic catalyst species, supported on an inorganic solid support; (b) subjecting said potential inorganic catalyst species to pre-determined reaction conditions in a single reactor in the presence of pre-determined reactants to provide a reaction products stream; (c) analyzing the reaction products stream; and if desired reaction products are present; (d) splitting said pool into at least two portions and repeating step (b) until a single catalyst mixture or multiple catalyst mixtures are identified which satisfy pre-determined criteria.

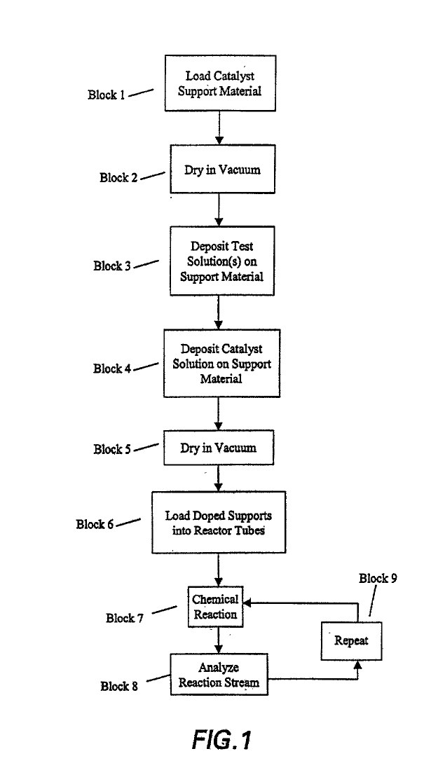

In one embodiment, FIG. 1 illustrates a high throughput doping of a catalyst support material. The catalyst support material is loaded (Block 1), dried under vacuum (Block 2), a test solution is deposited on the support material (Block 3), a catalyst solution is deposited on the support material (Block 4), the support material and solution are dried under vacuum (Block 5), the doped supports are loaded into reactor tubes (Block 6), a predetermined chemical reaction takes place (Block 7), and the reaction stream is analyzed for desired results (Block 8). The reaction may be repeated (Block 9) and then analyzed again (Block 8).

In this method, any solid support which is generally used in a heterogeneous catalysis reaction, either continuous or batch, may be utilized. Particularly preferred are solid supports such as alumina, MgCl₂, zeolites, silica, Al₂O₃, ZrO₂, TiO₂, SiO₂, carbon black, clays and diatomaceous earth. Preferred supports have about 100-200 m²/g surface area and 0.3-1.0 ml/g pore volume.

As noted above, the steps (b) through (d) can be repeated until a single catalyst mixture or multiple catalyst mixtures are identified which satisfy pre-determined criteria, ie., the presence of desired reaction product(s).

In this regard, potential reactions which can be analyzed and screened for catalytically active specie include oxidation and reduction reactions. More specifically, carbonylation, hydrogenation, de Nox, and methylation.

In a further embodiment, the solid support is in the form of pellets. In such cases where the desired catalytic reaction is conducted under conditions in which potential undesired side reactions include oxidative decomposition of the starting material, it is also possible to remove from the pool of potential catalysts a pellet which is visibly darkened in color, thus evidencing a charring or decomposition, i.e., pyrolysis of organic starting material(s). Likewise, in such circumstances it is also possible to select out those pellets which remain white or are at least lighter in color as those in or on which little or no such undesired oxidative decomposition occurred. Thus, an embodiment of the present invention involves visual separation of pellets based on appearance, e.g. dark/light.

The method of the present invention allows for rapid catalyst discovery and optimization using one reactor versus multiple reactors or several sequential reactions in one reactor.

Thus, in a further embodiment, there is provided a method for the rapid screening of catalytically active species, which includes the steps: (a) providing a gradient array of potential inorganic catalyst species, supported on an inorganic solid support, (b) subjecting said potential inorganic catalyst species to pre-determined reaction conditions in a single reactor in the presence of pre-determined reactants to provide a reaction products stream; and (c), analyzing the solid support visually or spectroscopically in order to determine whether oxidative decomposition of the starting materials has occurred, and if not, selecting out such support.

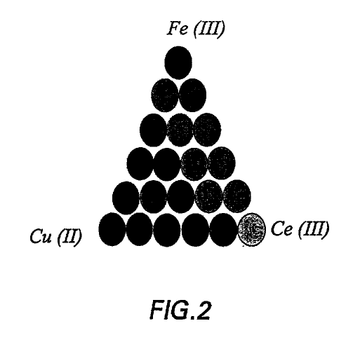

Referring to FIG. 2, in a further preferred embodiment, once a single catalyst mixture has been obtained, a gradient array can be prepared which provides variation of concentration for the individual components of such catalytic species.

Thus, in a further embodiment, there is provided the above method, further including the steps: (e) providing a gradient array of the single inorganic catalyst mixture or multiple catalyst mixtures; (f) subjecting said mixture(s) to pre-determined reaction conditions in a single reactor in the presence of pre-determined reactants to provide a reaction products stream; and (g), analyzing the reaction products stream; and splitting said array into at least two portions and repeating step (f) until a single catalyst mixture or multiple catalyst mixtures are identified which satisfy pre-determined criteria.

In this regard, pre-determined criteria may be the existence of desired product in the reaction products stream, either on an absolute basis or on a quantitative basis, e.g., where the pre-determined criteria would be a desired proportion and/or purity of one or more desired products.

Experimental Section

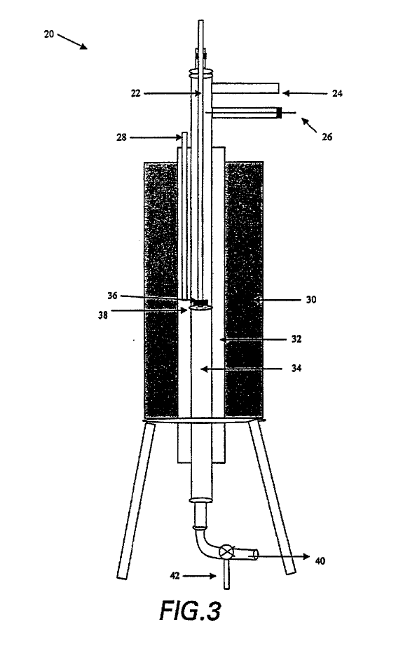

Stock solution of iron, copper and cerium nitrates (Alfa Aesar) were prepared at a concentration of 1 M. The 1 M solution was robotically mixed into single, binary and ternary mixtures at 20% concentration intervals using a four-probe liquid dispenser, see FIG. 2. Alumina pellets (Norton) with a pore volume of 1 ml/g were dried under vacuum for a minimum of two hours. Each of the metal mixtures were loaded onto a separate alumina pellet, the volume loaded was determined by the pore volume such that the doping was to incipient wetness. The doped alumina supports were dried at 100° C. under vacuum for at least two hours and loaded into the glass reactor 20 shown in FIG. 3.

Referring to FIG. 3, the glass reactor 20 included an inner thermocouple 22, a gas inlet 24, a 1/16″ needle (Swadge Loc fit) 26, an outer thermocouple 28, a tube furnace 30, a large outer quartz tube 32, an inner quartz reaction tube 34, a catalyst bed 36, a glass frit 38, a cold trap under vacuum 40, and a sample collection port 42.

The temperature of the reactor was ramped to 350° C. in air for one hour to decompose the nitrates. The reactor was cooled and a flow of 6% hydrogen in argon was applied. The temperature was ramped to 300° C. without the addition of reactants, to allow activation of the doped metal supports, and soaked for 1 hour. The addition of tetramethoxysilane (Aldrich) followed at a flow rate of 0.8 ml/min. Depending on experimental design, the reaction may be held isothermal with product samples collected at timed intervals, or the temperature may be varied with subsequent product sampling. Collected samples were analyzed for desired analytes using GC and GC-MS.

TABLE 1

| Library | Temp/C. | % D′ | % Q′ | % Q′ Dimer | % Q′ Trimer | Methanol | Acetone | Other |
|---|---|---|---|---|---|---|---|---|
| Cu, Fe, Ce | 350 | 0 | 96 | 3.8 | 0.15 | 0 | 0 | 0 |
| | 450 | 0 | 94.5 | 5 | 0.5 | 0 | 0 | 0 |
| | 550CT | 0 | 0 | 0 | 0 | 0 | 0 | dimethyl ether |
| Cu, Fe, Al | 250 | 0 | 57 | 3.8 | 0.2 | 1.8 | 35 | dimethoxydimethyl |
| | | | | | | | | methane 0.1% |
| | 300 | 0 | 89.6 | 4.5 | 0.4 | 0 | 5.2 | 0 |
| | 350 | 0 | 97 | 2.7 | 0.19 | 0 | 0 | 0 |
| | 350CT | 0 | 0 | 0 | 0 | 0 | yes | dimethyl ether |
| Cu, Fe, | 200 | 0 | 66.8 | 9 | 1.3 | 20 | 0 | 0 |
| Al/H2 10% | | | | | | | | |
| | 225 | 0 | 68.5 | 7.8 | 3 | 17.3 | 0 | 0 |
| | 250 | 0 | 95 | 4.16 | 0.4 | 0 | 0 | 0 |
| | CT | 0 | 0 | 0 | 0 | 0 | 0 | 0 |

Methods and systems for information dissemination have been described herein. These, and other variations, which will be appreciated by those skilled in the art, are within the intended scope of this invention as claimed below. As previously stated, detailed embodiments of the present invention are disclosed herein; however, it is to be understood that the disclosed embodiments are merely exemplary of the invention that may be embodied in various forms.

1. A method for the rapid screening of catalytically active species, which comprises the steps:
(a)providing a gradient array of potential inorganic catalyst species, supported on an inorganic solid support;
(b)subjecting said potential inorganic catalyst species to pre-determined reaction conditions in a single reactor in the presence of pre-determined reactants to provide a reaction products stream;
(c)analyzing the reaction products stream; and if desired reaction products are present; and
(d)splitting said pool into at least two portions and repeating step (b) until a single catalyst mixture or multiple catalyst mixtures are identified which satisfy pre-determined criteria.

2. The method of claim 1, wherein the solid support is selected from the group consisting of alumina, MgCl₂, zeolites, polymeric beads, silica, Al₂O₃, ZrO₂, TiO₂, SiO₂, carbon black, clays, minerals and diatomaceous earth.

3. The method of claim 1, wherein the potential catalyst species are comprised of elements selected from transition, lanthanide, actinide, and main group metals.

4. The method of claim 1, further comprising the step:
(e)providing a gradient array of the single catalyst mixture or multiple catalyst mixtures;
(f)subjecting said mixture(s) to pre-determined reaction conditions in a single reactor in the presence of pre-determined reactants to provide a reaction products stream; and
(g)analysis of the reaction products stream; and splitting said pool into at least two portions and repeating step (f) until a single catalyst mixture or multiple catalyst mixtures are identified which satisfy pre-determined criteria.

5. A method for the rapid screening of catalytically active species, which comprises the steps:
(a)providing a gradient array of potential inorganic catalyst species, supported on an inorganic solid support;
(b)subjecting said potential inorganic catalyst species to pre-determined reaction conditions in a single reactor in the presence of pre-determined reactants to provide a reaction products stream; and
(c)analyzing the solid support visually or spectroscopically in order to determine whether oxidative decomposition of the starting material(s) has occurred, and if not, selecting out such support.

6. The method of claim 5, wherein the solid support is selected from the group consisting of alumina, MgCl₂, zeolites, polymeric beads, silica, Al₂O₃, ZrO₂, TiO₂, SiO₂, carbon black, clays, minerals and diatomaceous earth.

7. The method of claim 5, wherein the potential catalyst species are comprised of elements selected from transition, lanthanide, actinide, and main group metals.

8. The method of claim 5, further comprising the step:
(d)providing a gradient array of the single catalyst mixture or multiple catalyst mixtures;
(e)subjecting said mixture(s) to pre-determined reaction conditions in a single reactor in the presence of pre-determined reactants to provide a reaction products stream; and
(f)analysis of the reaction products stream; and splitting [said pool] into at least two portions and repeating step (e) until a single catalyst mixture or multiple catalyst mixtures are identified which satisfy pre-determined criteria.
